(19) United States
(12) Patent Application Publication
(10) Pub. No.: US 2026/0325106 A1
(43) Pub. Date: Oct. 22, 2026

(54) Diffraction Tomography for Sound Speed Estimation and Aberration Correction in Medical Pulse-Echo Ultrasound Imaging Based on Migration Velocity Analysis

(71) Applicant: University of Rochester, Rochester, NY (US)

(72) Inventors: Rehman Ali, Rochester, NY (US); Nebojsa Duric, Rochester, NY (US)

(73) Assignee: University of Rochester, Rochester, NY (US)

(21) Appl. No.: 19/114,737

(22) PCT Filed: Oct. 22, 2023
(86) PCT No.: PCT/US2023/035656

§ 371 Date: Mar. 24, 2025

Related U.S. Application Data
(60) Provisional application No. 63/419,040, filed on Oct. 25, 2022.

(51) Int. Cl. A61B 8/00

(52) U.S. Cl. CPC A61B 8/5207; A61B 8/5269; A61B 8/565

(57) ABSTRACT

An improved system for medical pulse-echo ultrasound imaging based on migration velocity analysis is disclosed. The system uses diffraction tomography for sound speed estimation and aberration correction, and has a sending subsystem and a data acquisition subsystem, an ultrasound probe, a wave equation migration velocity analysis (WEMVA) based signal processing component that provides a reflectivity image and a sound speed map, and an output device configured to receive a reflectivity image and a sound speed map.

### CROSS REFERENCE TO RELATED PATENT APPLICATIONS

This application is a 371 entry of PCT/US2023/035656 filed on Oct. 22, 2023 entitled “Diffraction Tomography For Sound Speed Estimation And Aberration Correction In Medical Pulse-Echo Ultrasound Imaging Based On Migration Velocity Analysis” by Ali et al., and claims priority to U.S. Patent Application Ser. No. 63/419,040 filed on Oct. 25, 2022 entitled “Diffraction Tomography For Sound Speed Estimation And Aberration Correction In Medical Pulse-Echo Ultrasound Imaging Based On Migration Velocity Analysis” by Ali et al., the entire disclosures of which are incorporated herein by reference.

### STATEMENT REGARDING FEDERALLY SPONSORED RESEARCH OR DEVELOPMENT

### Technical Field

The present invention relates generally to Diffraction Tomography, and more particularly to Diffraction Tomography for Sound Speed Estimation and Aberration Correction in Medical Pulse-Echo Ultrasound Imaging Based on Migration Velocity Analysis.

### BACKGROUND ART

Distributed aberration correction in medical pulse-echo ultrasound relies on sound speed estimation, which currently is limited to ray based methods with estimated times of flight. In a heterogeneous sound speed medium such as that encountered during medical pulse-echo ultrasound imaging, the ability to estimate the true speed of sound in a heterogeneous medium such as the human body is important in order to optimally focus or correct for aberrations in the ultrasound image. Estimation of the speed of sound in ultrasound transmission tomography is often done with waveform inversion, a form of diffraction tomography where the error between measured and simulated channel data is used to estimate the speed of sound. However, sound speed estimation in pulse-echo ultrasound relies on reflections rather than the transmission of ultrasound through tissue and has largely employed ray based methods with estimated times of flight. Unfortunately, ray based approaches are often unreliable in-vivo where a simple time of flight model cannot account for heterogeneous backscatter and wavefront splitting. Wave equation migration velocity analysis, a form of diffraction tomography that uses the discrepancy between reflections imaged at different angles to estimate the speed of sound, has been shown to overcome the limitation of ray tomography in the field of seismology; however, such techniques have heretofore not been modified, developed or applied to medical pulse echo ultrasound, and in particular, to hand-held ultrasound. Improvements to sound speed estimation from the current status quo would provide for breakthrough improvements in image quality. What is therefore needed is a system for ultrasound imaging that has improved sound speed estimation and aberration correction.

### DISCLOSURE OF THE INVENTION

In accordance with the present invention, there is provided an improved system for ultrasound imaging comprising an ultrasound system comprising a sending subsystem and a data acquisition subsystem; an ultrasound probe operatively connected to the ultrasound system; a wave equation migration velocity analysis based signal processing component that provides a reflectivity image and a sound speed map; and an output device configured to receive a reflectivity image and a sound speed map.

In one embodiment of the present invention, the wave equation migration velocity analysis-based signal processing component comprises a shot-profile migration algorithm, a wave equation migration velocity analysis (WEMVA) algorithm, and a sound speed map output from the wave equation migration velocity analysis (WEMVA) algorithm and input to the shot-profile migration algorithm.

In one embodiment of the present invention, the wave equation migration velocity analysis (WEMVA) based signal processing component further comprises a coherent compounding algorithm configured to receive reconstructed images from the shot-profile migration algorithm and generate a reflectivity image.

In one embodiment of the present invention, the wave equation migration velocity analysis (WEMVA) algorithm is ray based.

In one embodiment of the present invention, the output device may be a display, a digital storage device, or a printer.

In one embodiment of the present invention, the improved system for ultrasound imaging further comprises a network interface operatively connected to the output device.

In one embodiment of the present invention, the wave equation migration velocity analysis (WEMVA) based signal processing component is configured to receive and process received data and send metadata from the ultrasound system.

In one embodiment of the present invention, a wave equation migration velocity analysis based (WEMVA) signal processing system is disclosed that comprises a shot-profile migration algorithm, a wave equation migration velocity analysis (WEMVA) algorithm, and a sound speed map output from the wave equation migration velocity analysis (WEMVA) algorithm and input to the shot-profile migration algorithm.

In one embodiment of the present invention, the wave equation migration velocity analysis (WEMVA) based signal processing system further comprises a coherent compounding algorithm configured to receive reconstructed images from the shot-profile migration algorithm and generate a reflectivity image.

In one embodiment of the present invention, the wave equation migration velocity analysis (WEMVA) based signal processing system is ray based.

A method for iterative image refinement and sound speed tissue characterization is disclosed, the method comprising the steps of: acquiring medical ultrasound pulsed-echo data from an ultrasound data acquisition system; applying a shot-profile migration algorithm to reconstruct images from the acquired data; applying a wave equation migration velocity analysis (WEMVA) algorithm to the reconstructed images to update a sound speed map; and providing updated sound speed map data to the shot-profile migration algorithm.

In one embodiment of the present invention, the method further comprises the step of providing the updated sound speed map to an output device.

further comprising the steps of:

In one embodiment of the present invention, the method further comprises the steps of providing reconstructed images from the shot-profile migration algorithm to a coherent compounding algorithm; and generating a reflectivity image from the reconstructed images provided to the shot-profile migration algorithm.

In one embodiment of the present invention, the method further comprises the step of providing the reflectivity image to an output device.

further comprising the step of:

In one embodiment of the present invention, the method further comprises the step of providing the updated sound speed map and the reflectivity image to an output device.

In one embodiment of the present invention, the output device is connected to a network interface.

In one embodiment of the present invention, the output device may be a display, a digital storage device, or a printer.

In one embodiment of the present invention, the medical ultrasound pulsed-echo data comprises receive data and send metadata.

In one embodiment of the present invention, the method further comprises the step of: providing the updated sound speed map and the reflectivity image to a network.

In one embodiment of the present invention, the wave equation migration velocity analysis (WEMVA) algorithm is ray based.

The foregoing has been provided by way of introduction, and is not intended to limit the scope of the invention as described by this specification and the attached drawings.

### BRIEF DESCRIPTION OF THE DRAWINGS

The invention will be described by reference to the following drawings, in which like numerals refer to like elements, and in which:

FIG. 1 depicts a block diagram of an improved system for ultrasound imaging in accordance with one embodiment of the present invention;

FIG. 2 is a flowchart depicting a method of the present invention;

FIG. 3 is a flowchart depicting a method for iterative image refinement and sound speed tissue characterization in accordance with one embodiment of the present invention;

FIG. 4 depicts an angular spectrum method in polar coordinates for curvilinear arrays in accordance with one embodiment of the present invention;

FIG. 5 depicts pulse-echo ultrasound imaging based on the time-domain cross-correlation of transmit and receive wavefields;

FIG. 6 depicts sound speed estimation and phase aberration correction based on WEMVA applied to reconstructed images;

FIG. 7 is a schematic diagram of the time of flight from transmitter to image point to receiver;

FIG. 8 depicts slowness gradients for the pulse-echo reconstructions of a point target; and

FIGS. 9 and 10 depict two different simulated abdominal imaging cases before aberration correction and after corrections.

The present invention will be described in connection with a preferred embodiment, however, it will be understood that there is no intent to limit the invention to the embodiment described. On the contrary, the intent is to cover all alternatives, modifications, and equivalents as may be included within the spirit and scope of the invention as defined by this specification, drawings and claims attached hereto.

### BEST MODE FOR CARRYING OUT THE INVENTION

Phase aberration is one the key sources of image degradation in handheld B-mode ultrasound imaging. Sound speed heterogeneities create phase aberrations in the image by inducing additional tissue-dependent delays and diffractive effects that conventional beamforming does not incorporate. For this reason, and in accordance with the present invention, the Fourier split-step angular spectrum method is used to simulate pressure fields in a spatially varying sound speed medium. B-mode ultrasound images are then created using a seismic imaging technique known as shot-profile migration. This Fourier split step angular spectrum method of the present invention may be implemented for either a linear or a curvilinear array.

The shot profile migration technique reconstructs the ultrasound image by correlating the transmitted and back-propagated received signals. Because shot profile migration is parameterized by the sound speed in the medium via the Fourier split-step angular spectrum, the image reconstructed by shot profile migration may be optimized with respect to sound speed to simultaneously estimate the sound speed profile in the medium and correct aberrations in the image. This approach describes a form of diffraction tomography known as wave-equation migration velocity analysis (WEMVA) in the field of seismic imaging.

For a more thorough understanding of the present invention and the various embodiments described and envisioned herein, reference is now made to the Figures and accompanying descriptions thereof.

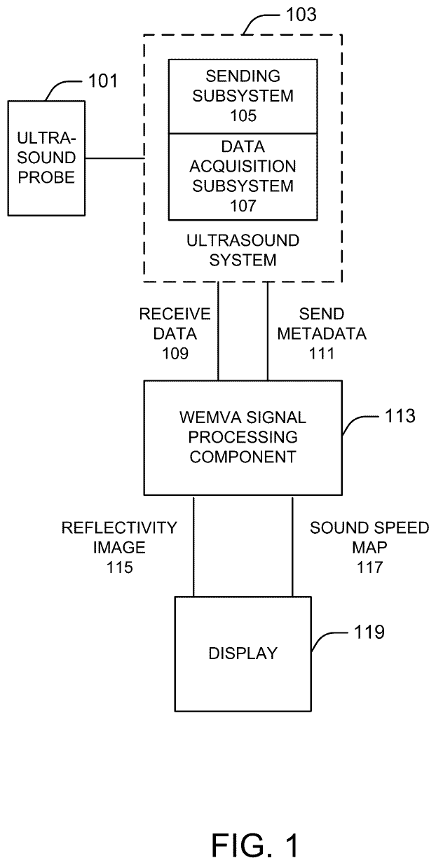

FIG. 1 depicts a block diagram of an improved system for ultrasound imaging in accordance with one embodiment of the present invention. An ultrasound system 103 can be seen with an ultrasound probe 101 connected therewith. The ultrasound system comprises a sending subsystem 105 that provides sound waves and a data acquisition subsystem 107 that receives the returned sound waves. The data acquisition subsystem 107 has a processor, memory and access to computer readable media. Additionally, the sending subsystem may also have a processor, memory, and access to computer readable media. The ultrasound system 103 provides both receive data 109 and send metadata 111 to a Wave Equation Migration Velocity Analysis (WEMVA) signal processing component 113. The WEMVA signal processing component includes software, the mathematical details of which are further described herein and whose output includes a reflectivity image 115 that may be provided to a display for visual examination, another computer or data storage device, a printer, a network interface, and the like. The WEMVA signal processing component 113 also has a processor, memory and access to computer readable media, and provides a reflectivity image 115 and a sound speed map 117 to an output device such as a display 119, a digital storage device, a printer, or the like, for interpretation by medical personnel. In some embodiments of the present invention, a network interface is operatively connected to the output device. The display 119 may also provide the reflectivity image 115 and the sound speed map 117 to a network, a computer program, a database, or the like. In some embodiments of the present invention, the reflectivity image 115 and the sound speed map 117 are provided to a downstream system for further processing, analysis, or the like.

The wave equation migration velocity analysis (WEMVA) based signal processing component 113 comprises a processor, memory, and access to computer readable media, and further comprises a shot-profile migration algorithm, a wave equation migration velocity analysis (WEMVA) algorithm, and a sound speed map output from the wave equation migration velocity analysis (WEMVA) algorithm and input to the shot-profile migration algorithm.

In some embodiments, the wave equation migration velocity analysis-based signal processing component further comprises a coherent compounding algorithm configured to receive reconstructed images from the shot-profile migration algorithm and generate a reflectivity image.

In some embodiments, the wave equation migration velocity analysis based signal processing component is configured to receive and process received data and send metadata from the ultrasound system.

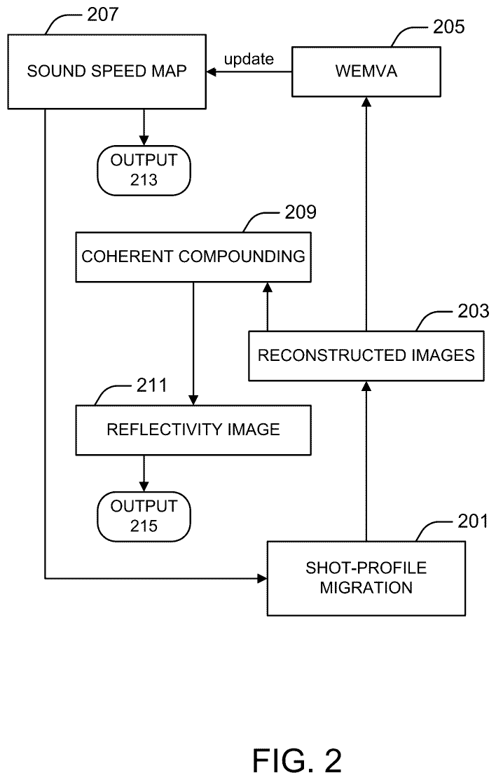

FIG. 2 is a flowchart depicting a method of the present invention. Shot profile migration is used in 201 to create a reflectivity image based on the sound speed profile. Reconstructed images 203 are then sent to Wave Equation Migration Velocity Analysis (WEMVA) in step 205, which are then used to update a sound speed map 207 which can then provide an output 213 of the sound speed map 207. The reconstructed images 203 are also sent to a coherent compounding step 209 which are then used to create a reflectivity image 211 and a related output 215. The created or updated sound speed map 207 is then used as a basis for a further shot profile migration step 201.

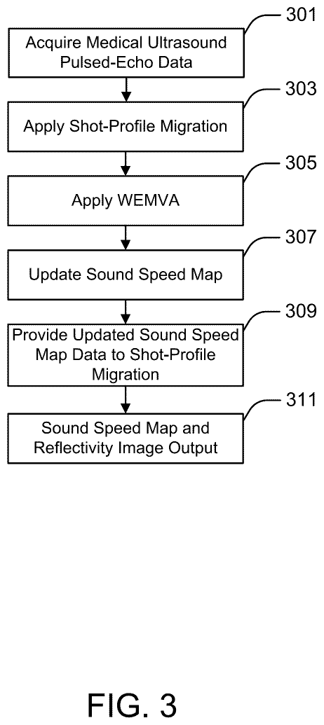

FIG. 3 is a flowchart depicting a method for iterative image refinement and sound speed tissue characterization in accordance with one embodiment of the present invention. The method described is implemented on a computer having a processor, memory and access to computer readable media.

The method comprises the steps of acquiring medical ultrasound pulsed-echo data from an ultrasound data acquisition system in step 301 (see FIG. 3), applying a shot-profile migration algorithm to reconstruct images from the acquired data in step 303, applying a wave equation migration velocity analysis (WEMVA) algorithm in step 305 to the reconstructed images to update a sound speed map in step 307, providing an updated sound speed map data to the shot-profile migration algorithm in step 309, and creating or updating a sound speed map and reflectivity image output in step 311.

The method may also include the step of providing the updated sound speed map to an output device.

The method may also include the steps of providing reconstructed images from the shot-profile migration algorithm to a coherent compounding algorithm, and generating a reflectivity image from the reconstructed images provided to the shot-profile migration algorithm.

The method may also include the step of providing the reflectivity image to an output device.

The method may also include the step of providing the updated sound speed map and the reflectivity image to an output device.

The output device may be a display, a digital storage device, a printer, a network or a network interface, or the like.

The medical ultrasound pulsed-echo data comprises receive data and send metadata.

The method may also include the step of providing the updated sound speed map and the reflectivity image to a network.

Various methods of the present invention will now be described herein.

### A. Fourier Split-Step Angular Spectrum Method for Linear Arrays

Ultrasonic wave-fields p(x,z,f) as a function of location (x,z) and frequency f can be propagated from a depth of z to z+Δz using the Fourier split-step angular spectrum method:

$$p ( x , z + Δ z , f ) = W ( x ) {S}_{\pm } ( x , z , f ) {F}_{{k}_{x \to x}}^{- 1} \{ {H}_{\pm } ( {k}_{x} , z , f ) {F}_{x \to {k}_{x}} [ p ( x , z , f ) ] \} , \qquad ( 1 )$$

$${H}_{\pm } ( {k}_{x} , z , f ) = exp ( \pm j 2 {π ( {( \frac{f}{\overline{c} ( z )} )}^{2} - {k}_{x}^{2} )}^{\frac{1}{2}} Δ z ) , \qquad ( 2 )$$

$${S}_{\pm } ( x , z , f ) = exp ( \pm \frac{j 2 π f Δ z}{{c}_{r e s} ( x , z )} ) , \qquad ( 3 )$$

$$\frac{1}{\overline{c} ( z )} = {\int }_{- L / 2}^{L / 2} \frac{d x}{c ( x , z )} , \qquad ( 4 )$$

$$\frac{1}{{c}_{r e s} ( x , z )} = \frac{1}{c ( x , z )} - \frac{1}{\overline{c} ( z )} , \qquad ( 5 )$$

where

$${F}_{x \to {k}_{x}} and {F}_{{k}_{x} \to x}^{- 1}$$

are the forward and inverse Fourier transforms in the lateral dimension (x), and c(x,z) is the speed of sound in the medium as a function of (x,z), and L is the lateral length of the computational domain over which the angular spectrum method is applied.

### B. Fourier Split-Step Polar-Coordinate Angular Spectrum Method for Curvilinear Arrays

Ultrasonic wave-fields p(θ,r,f) as a function of location (θ,r) and frequency f can be propagated from a radial range of r=rᵢ to r=r₂ using the polar coordinate form of angular spectrum method (FIG. 1) [2]:

$$p ( θ , {r}_{2} , f ) = W ( θ ) {S}_{\pm } ( θ , {r}_{1} , {r}_{2} , f ) {F}_{{k}_{θ} \to θ}^{- 1} \{ {H}_{\pm } ( {k}_{θ} , {r}_{1} , {r}_{2} , f ) {F}_{θ \to {k}_{θ}} [ p ( θ , {r}_{1} , f ) ] \} , \qquad ( 6 )$$

$${H}_{\pm } ( {k}_{θ} , {r}_{1} , {r}_{2} , f ) = exp ( \pm j 2 π {\vert {k}_{θ} \vert [ {( {( \frac{f r}{{k}_{θ} \overline{c} ( r )} )}^{2} - 1 )}^{\frac{1}{2}} - {arctan ( {( \frac{f r}{{k}_{θ} \overline{c} ( r )} )}^{2} - 1 )}^{\frac{1}{2}} ]}_{r = {r}_{1}}^{r = {r}_{2}} ) , \qquad ( 7 )$$

$${S}_{\pm } ( θ , {r}_{1} , {r}_{2} , f ) = exp ( \pm \frac{j 2 π f ( {r}_{2} - {r}_{1} )}{{c}_{r e s} ( θ , \frac{{r}_{2} + {r}_{1}}{2} )} ) , \qquad ( 8 )$$

$$\frac{1}{\overline{c} ( r )} = {\int }_{- {θ}_{max} / 2}^{{θ}_{max} / 2} \frac{d θ}{c ( θ , r )} \qquad ( 9 )$$

$$\frac{1}{{c}_{r e s} ( θ , r )} = \frac{1}{c ( θ , r )} - \frac{1}{\overline{c} ( r )} , \qquad ( 10 )$$

where

$${F}_{θ \to {k}_{θ}} and {F}_{{k}_{θ} \to θ}^{- 1}$$

are the forward and inverse Fourier transforms in the angular dimension (θ), and c(θ,r) is the speed of sound in the medium as a function of (θ,r), and θₘₐₓ is the angular coverage of the computational domain over which the angular spectrum method is applied.

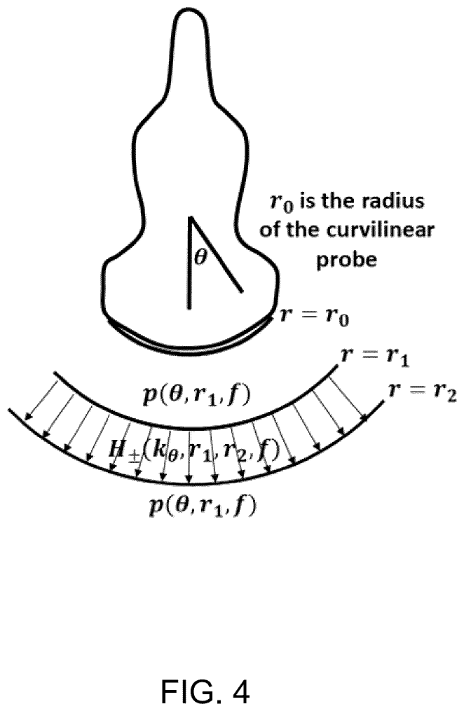

FIG. 4 depicts the angular spectrum method in polar coordinates for curvilinear arrays, as described by way of equations 1 and 2 above.

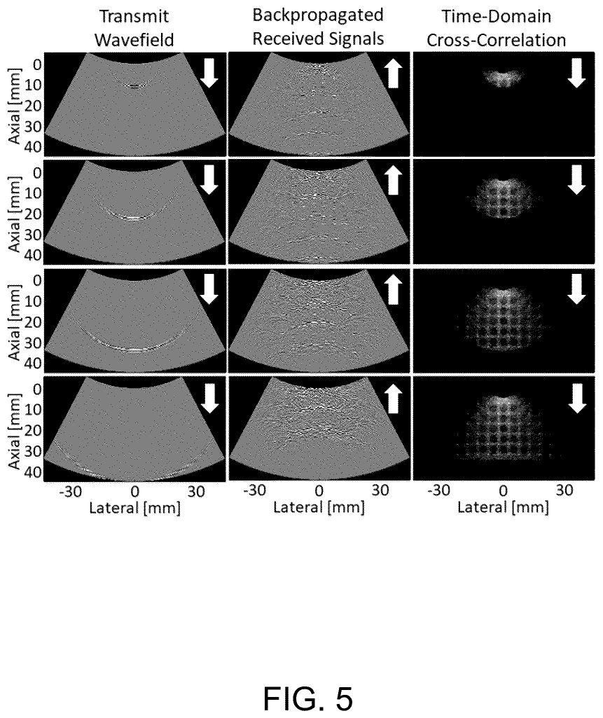

FIG. 5 depicts pulse-echo ultrasound imaging based on the time-domain cross-correlation of transmit and receive wavefields.

### C. Shot-Profile Migration: Imaging by Correlation of Transmit and Receive Wavefields

Pulse-echo ultrasound images can be created by propagating transmit and receive wavefields pₜₓ₍ᵢ₎(x,z,f) and pᵣₓ₍ᵢ₎(x,z,f) based on the transmit sequence and receive channel data collected from each transmit element i=1, . . . , Nₜₓ. Partial images Iᵢ(x,z) prior to amplitude detection can be formed based on the following equations:

$${I}_{i} ( x , z ) = {\int }_{0}^{∞} {P}_{tx ( i )}^{*} ( x , z , f ) {p}_{r x ( i )} ( x , z , f ) df , \qquad ( 11 )$$

$${p}_{tx ( i )} ( x , z + Δ z , f ) = W ( x ) {S}_{-} ( θ , r , f ) {F}_{{k}_{θ} \to θ}^{- 1} \{ {H}_{-} ( {k}_{x} , z , f ) {F}_{θ \to {k}_{θ}} [ {p}_{tx ( i )} ( θ , r , f ) ] \} , \qquad ( 12 )$$

$${p}_{r x ( i )} ( x , z + Δ z , f ) = W ( x ) {S}_{+} ( x , z , f ) {F}_{{k}_{θ} \to θ}^{- 1} \{ {H}_{+} ( {k}_{x} , z , f ) {F}_{θ \to {k}_{θ}} [ {p}_{r x ( i )} ( x , z , f ) ] \} . \qquad ( 13 )$$

Shot profile migration is functionally the same for curvilinear arrays except that the polar coordinate form of the angular spectrum method is used instead:

$${I}_{i} ( θ , r ) = {\int }_{0}^{∞} {P}_{tx ( i )}^{*} ( θ , r , f ) {p}_{r x ( i )} ( θ , r , f ) df , \qquad ( 14 )$$

$${p}_{tx ( i )} ( θ , {r}_{2} , f ) = W ( θ ) {S}_{-} ( θ , {r}_{1} , {r}_{2} , f ) {F}_{{k}_{θ} \to θ}^{- 1} \{ {H}_{-} ( {k}_{θ} , {r}_{1} , {r}_{2} , f ) {F}_{θ \to {k}_{θ}} [ {p}_{tx ( i )} ( θ , {r}_{1} , f ) ] \} , \qquad ( 15 )$$

$${p}_{r x ( i )} ( θ , {r}_{2} , f ) = W ( θ ) {S}_{+} ( θ , {r}_{1} , {r}_{2} , f ) {F}_{{k}_{θ} \to θ}^{- 1} \{ {H}_{+} ( {k}_{θ} , {r}_{1} , {r}_{2} , f ) {F}_{θ \to {k}_{θ}} [ {p}_{r x ( i )} ( θ , {x}_{1} , f ) ] \} , \qquad ( 16 )$$

FIG. 5 visually demonstrates a time-domain implementation of shot-profile migration on a curvilinear array. This approach is fundamentally the same regardless of the coordinate system used.

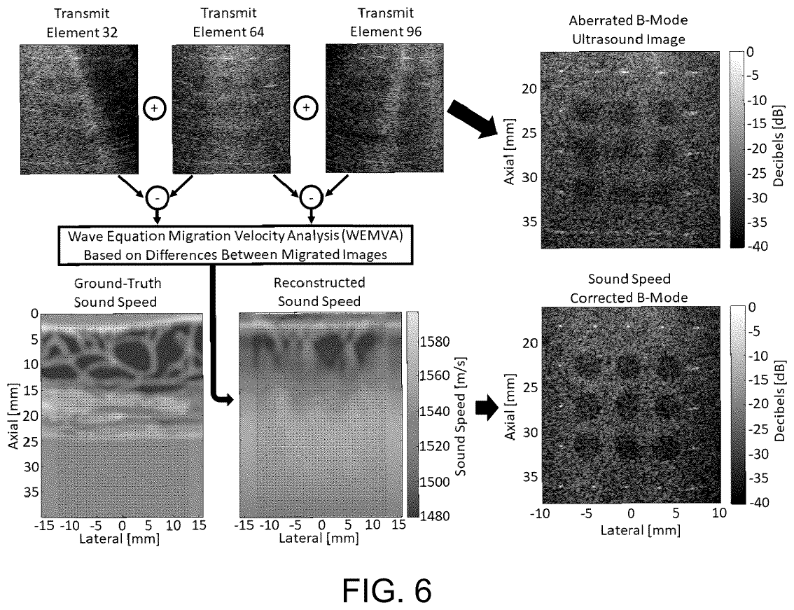

FIG. 6 depicts sound speed estimation and phase aberration correction based on WEMVA applied to reconstructed images.

### D. Wave-Equation Migration Velocity Analysis (WEMVA)

The following vectorized notation for shot profile migration applies to all forms of the angular spectrum method regardless of the coordinate system used:

$${\vec{I}}_{i} ( \vec{s} ) = {\sum }_{f} {\vec{p}}_{Rx , i}^{*} ( \vec{c} , f ) ∘ {\vec{p}}_{Tx , i} ( c , f ) , \qquad ( 17 )$$

where ∘ is a point-wise multiplication of the transmit and receive wavefields \vec{p}_{Tx,i} and \vec{p}_{Rx,i}, the summation over f replaces the integrals used in (11) and (14), and \vec{I}ᵢ is the image parameterized by the sound speed \vec{c} in the medium. The derivative of \vec{I}ᵢ with respect to \vec{c} is:

$$\frac{\partial {\vec{I}}_{i} ( \vec{c} )}{\partial \vec{c}} = {\sum }_{f} \frac{\partial {\vec{p}}_{Rx , i}^{*} ( \vec{c} , f )}{\partial \vec{c}} ∘ {\vec{p}}_{Tx , i} ( \vec{c} , f ) + {\vec{p}}_{Rx , i}^{*} ( \vec{c} , f ) ∘ \frac{\partial {\vec{p}}_{T x i} ( \vec{c} , f )}{\partial \vec{c}} , \qquad ( 18 )$$

The following provides a linearization of Iᵢ with respect to perturbations in sound speed Δ\vec{c}:

$${\vec{I}}_{i} ( \vec{c} + Δ \vec{c} ) = {\vec{I}}_{i} ( \vec{c} ) + \frac{\partial {\vec{I}}_{i} ( \vec{c} )}{\partial \vec{c}} Δ \vec{c} \approx {\vec{I}}_{i + \frac{1}{2}} ( \vec{c} ) + \frac{\partial {\vec{I}}_{i} ( \vec{c} )}{\partial \vec{c}} Δ \vec{c} , \qquad ( 19 )$$

$${\vec{I}}_{i + 1} ( \vec{c} + Δ \vec{c} ) = {\vec{I}}_{i + 1} ( \vec{c} ) + \frac{\partial {\vec{I}}_{i + 1} ( \vec{c} )}{\partial \vec{c}} Δ \vec{c} \approx {\vec{I}}_{i + \frac{1}{2}} ( \vec{c} ) + \frac{\partial {\vec{I}}_{i + 1} ( \vec{c} )}{\partial \vec{c}} Δ \vec{c} , \qquad ( 20 )$$

The short-lag approximation

$${\vec{I}}_{i} ( \vec{c} ) \approx {\vec{I}}_{i + \frac{1}{2}} ( \vec{c} ) \approx {\vec{I}}_{i + 1} ( \vec{c} )$$

assumes that images from neighboring transmit events are sufficiently similar that any discrepancy between them is the result of errors in c:

$${\vec{I}}_{i + 1} ( \vec{c} + Δ \vec{c} ) - {\vec{I}}_{i} ( \vec{c} + Δ \vec{c} ) \approx ( \frac{\partial {\vec{\vec{I}}}_{i + 1} ( \vec{c} )}{\partial \vec{c}} - \frac{\partial \vec{{\vec{I}}_{i}} ( \vec{c} )}{\partial \vec{c}} ) Δ \vec{c} . \qquad ( 21 )$$

We minimize the discrepancy between image from neighboring transmit events to estimate the spatial profile of sound speed \vec{c} and correct aberration in the final image obtained by coherent compounding \vec{I}_{final}=Σᵢ\vec{I}ᵢ.

The objective function for WEMVA and its gradient is:

$$J ( Δ \vec{c} ) = \frac{1}{2} {\sum }_{i} {‖ ( \frac{\partial \vec{{\vec{I}}_{i + 1}} ( \vec{c} )}{\partial \vec{c}} - \frac{\partial \vec{{\vec{I}}_{i}} ( \vec{c} )}{\partial \vec{c}} ) Δ \vec{c} - ( {\vec{I}}_{i + 1} - {\vec{I}}_{i} ) ‖}_{2}^{2} , \qquad ( 22 )$$

$${∇}_{Δ \vec{c}} J ( Δ \vec{c} ) \approx {\sum }_{i} {( \frac{\partial {\vec{I}}_{i + 1} ( \vec{c} )}{\partial \vec{c}} - \frac{\partial {\vec{I}}_{i} ( \vec{c} )}{\partial \vec{c}} )}^{H} ( ( \frac{\partial {\vec{I}}_{i + 1} ( \vec{c} )}{\partial \vec{c}} - \frac{\partial {\vec{I}}_{i} ( \vec{c} )}{\partial \vec{c}} ) Δ \vec{c} - ( {\vec{I}}_{i + 1} - {\vec{I}}_{i} ) ) . \qquad ( 23 )$$

The conjugate gradient algorithm based on the gradient of the objective function is used to iteratively estimate the sound speed profile \vec{c} and optimally align images \vec{I}ᵢ prior to coherent compounding. The schematic for the operation of WEMVA is shown in FIG. 6.

In some embodiments of the present invention, in order reduce computational costs related to such areas as memory and computational limitations, the Wave Equation Migration Velocity Analysis (WEMVA) approach described herein may be modified using a ray-based approach. Such a ray-based Wave Equation Migration Velocity Analysis (WEMVA) is further described as follows.

### A. Delay-and-Sum Beamforming Model

We first begin with a multistatic synthetic aperture dataset \tilde{p}ₘₙ(t) indexed by single-element transmit m and receiver n as a function of time t. For an N-element array, let τₗₖ(s) refer to the time delay computed as the line integral of slowness (vectorized as \vec{s}) over the path from element l to an image point

$${\vec{x}}_{k}^{i m} .$$

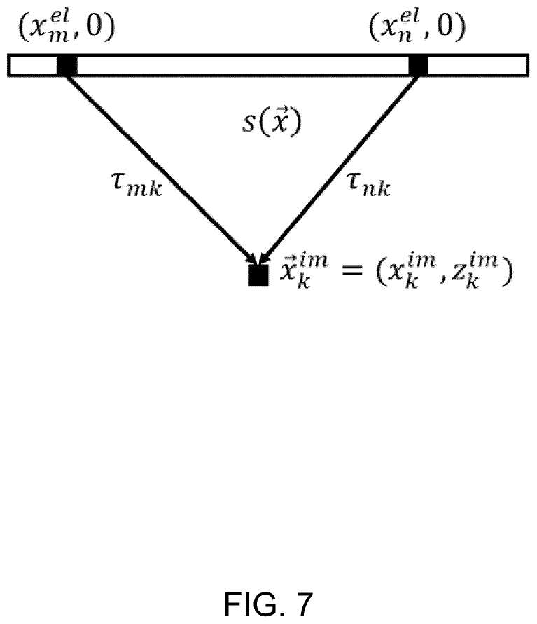

FIG. 7 is a schematic representation for the time-of-flight of the ultrasound wave from transmitter to image point back to the receiver. The time of flight is the line integral of slowness over the path. As per FIG. 7, we can focus the full matrix of transmit and receive signals at each image point

$${\vec{x}}_{k}^{i m}$$

by applying focusing delays:

$${p}_{mn} ( {\vec{x}}_{k}^{im} ; \vec{s} ) = {\tilde{p}}_{mn} ( {τ}_{mk} ( \vec{s} ) + {τ}_{nk} ( \vec{s} ) ) .$$

The result can then be summed over the receivers to obtain complex-valued images for each single-element transmit:

$${I}_{m} ( {\vec{x}}_{k}^{im} ; \vec{s} ) = {\sum }_{n = 1}^{N} {p}_{mn} ( {\vec{x}}_{k}^{im} ; \vec{s} ) .$$

Although WEMVA uses shot-profile migration to produce

$${I}_{m} ( {\vec{x}}_{k}^{im} ; \vec{s} ) ,$$

the above equations describe how delay-and-sum beamforming can be used to produce the same

$${I}_{m} ( {\vec{x}}_{k}^{im} ; \vec{s} )$$

images. Replacing shot-profile migration with a delay-and-sum beamformer yields ray-centric implementation of WEMVA. The least-squares objective function used in WEMVA is

$$J ( \vec{s} ) = \frac{1}{2} {\sum }_{k} {\sum }_{m = 1}^{N - 1} {\vert {R}_{m} ( {\vec{x}}_{k}^{im} ; \vec{s} ) \vert}^{2} ,$$

where

$${R}_{m} ( {\vec{x}}_{k}^{im} ; \vec{s} ) = {I}_{m + 1} ( {\vec{x}}_{k}^{im} ; \vec{s} ) - {I}_{m} ( {\vec{x}}_{k}^{im} ; \vec{s} )$$

are the differences between images from adjacent transmitters.
B. Gradient with Respect to Slowness

The gradient of the WEMVA objective function with respect to slowness is:

$$\frac{\partial J}{\partial \vec{s}} = Re \{ {\sum }_{k} {\sum }_{m = 1}^{N - 1} {( \frac{\partial {R}_{m} ( {\vec{x}}_{k}^{im} )}{\partial \vec{s}} )}^{H} {R}_{m} ( {\vec{x}}_{k}^{im} ) \}$$

where

$$\frac{\partial {R}_{m} ( {\vec{x}}_{k}^{im} )}{\partial \vec{s}} = \frac{\partial {I}_{m + 1} ( {\vec{x}}_{k}^{im} )}{\partial \vec{s}} - \frac{\partial {I}_{m} ( {\vec{x}}_{k}^{im} )}{\partial \vec{s}}$$

(⋅)^{H} is the Hermitian or conjugate transpose. Although WEMVA computes

$$\frac{\partial {I}_{m} ( {\vec{x}}_{k}^{im} )}{\partial \vec{s}}$$

based on the shot profile migration process, we now describe the same computation in terms of the delay-and-sum beamforming model:

$$\frac{\partial {I}_{m} ( {\vec{x}}_{k}^{im} )}{\partial \vec{s}} = {\sum }_{n = 1}^{N} \frac{\partial {p}_{mn}}{\partial \vec{s}} ( {\vec{x}}_{k}^{im} ; \vec{s} ) = {\sum }_{n = 1}^{N} [ \frac{\partial {\tilde{p}}_{mn}}{\partial t} ( {τ}_{mk} ( \vec{s} ) + {τ}_{nk} ( \vec{s} ) ) ] ( \frac{\partial {τ}_{mk}}{\partial \vec{s}} + \frac{\partial {τ}_{nk}}{\partial \vec{s}} )$$

where

$$[ \frac{\partial {\tilde{p}}_{mn}}{\partial t} ( {τ}_{mk} ( \vec{s} ) + {τ}_{nk} ( \vec{s} ) ) ]$$

is the application of focusing delays to the time-derivative of the multistatic channel data, and

$$\frac{\partial {τ}_{lk}}{\partial \vec{s}}$$

is the matrix of path lengths over each pixel of the slowness map from element l to an image point

$${\vec{x}}_{k}^{im} .$$

Note that

$$\frac{\partial {τ}_{mk}}{\partial \vec{s}} and \frac{\partial {τ}_{nk}}{\partial \vec{s}}$$

correspond to transmit and receive paths, respectively.

Conventional travel-time tomography would essentially assume that each single-element transmit image involves the same receive paths back to the transducer; therefore, any difference between images

$${I}_{m} ( {\vec{x}}_{k}^{im} ; \vec{s} ) and {I}_{m + 1} ( {\vec{x}}_{k}^{im} ; \vec{s} )$$

should be entirely due to delays along the transmit paths back to the transducer because all receive paths should cancel.

However, in this ray-centric approach to WEMVA, receive paths do not cancel when computing

$${R}_{m} ( {\vec{x}}_{k}^{im} ; \vec{s} ) .$$

There are nonzero contributions of

$$\{ \frac{\partial {\tilde{p}}_{m + 1 , n}}{\partial t} ( {τ}_{m + 1 , k} + {τ}_{nk} ) - \frac{\partial {\tilde{p}}_{mn}}{\partial t} ( {τ}_{mk} + {τ}_{nk} ) \}$$

along receive paths

$$\frac{\partial {τ}_{nk}}{\partial \vec{s}} .$$

This demonstrates the engagement of both transmit and receive paths back to the transducer in WEMVA.

### C. Grouping Terms by Ray Path

For m=1, . . . , N−1 and n=1, . . . , N, we define auxiliary variables

$${u}_{mn} ( {\vec{x}}_{k}^{im} ) and {v}_{mn} ( {\vec{x}}_{k}^{im} ) :$$

$${u}_{m + 1 , n} ( {\vec{x}}_{k}^{im} ) = [ \frac{\partial {\tilde{p}}_{m + 1 , n}^{*}}{\partial t} ( {τ}_{m + 1 , k} + {τ}_{nk} ) ] {R}_{m} ( {\vec{x}}_{k}^{im} ) ,$$

$${v}_{mn} ( {\vec{x}}_{k}^{im} ) = [ \frac{\partial {\tilde{p}}_{mn}^{*}}{\partial t} ( {τ}_{mk} + {τ}_{nk} ) ] {R}_{m} ( {\vec{x}}_{k}^{im} ) ,$$

where the * in the superscripts refers to complex conjugation. We can then find a single value

$${b}_{l} ( {\vec{x}}_{k}^{im} )$$

to backproject along the path from each image point

$${\vec{x}}_{k}^{im}$$

to each element l:

$${b}_{l} ( {\vec{x}}_{k}^{im} ) = \{ \begin{matrix}Re \{ - {\sum }_{n = 1}^{N} {v}_{1 , n} ( {\vec{x}}_{k}^{im} ) + {\sum }_{m = 2}^{N} {u}_{m , 1} ( {\vec{x}}_{k}^{im} ) - {\sum }_{m = 1}^{N - 1} {v}_{m , 1} ( {\vec{x}}_{k}^{im} ) \} & l = 1 \\ Re \{ {\sum }_{n = 1}^{N} {u}_{N , n} ( {\vec{x}}_{k}^{im} ) + {\sum }_{m = 2}^{N} {u}_{m , N} ( {\vec{x}}_{k}^{im} ) - {\sum }_{m = 1}^{N - 1} {v}_{m , N} ( {\vec{x}}_{k}^{im} ) \} & l = N \\ Re \{ {\sum }_{n = 1}^{N} ( {u}_{ln} ( {\vec{x}}_{k}^{im} ) - {v}_{ln} ( {\vec{x}}_{k}^{im} ) ) + {\sum }_{m = 2}^{N} {u}_{m , N} ( {\vec{x}}_{k}^{im} ) - {\sum }_{m = 1}^{N - 1} {v}_{m , N} ( {\vec{x}}_{k}^{im} ) \} & otherwise\end{matrix} .$$

The gradient of the WEMVA objective function can then be computed as

$$\frac{\partial J}{\partial \vec{s}} = Re \{ {\sum }_{k} {\sum }_{l = 1}^{N} {b}_{l} ( {\vec{x}}_{k}^{im} ) {( \frac{\partial {τ}_{lk}}{\partial \vec{s}} )}^{T} \} .$$

Therefore, the gradient of the WEMVA objective function may be computed by backprojecting

$${b}_{l} ( {\vec{x}}_{k}^{im} ) .$$

Any adjoint-state method can be used to achieve this. If shot profile migration were used instead of a delay-and-sum beamformer, an equivalent set of computations would result in the

$${b}_{l} ( {\vec{x}}_{k}^{im} )$$

values; the main change would be that instead of backprojecting

$${b}_{k} ( {\vec{x}}_{k}^{im} )$$

along ray paths, shot-profile migration would involve backprojecting

$${b}_{l} ( {\vec{x}}_{k}^{im} )$$

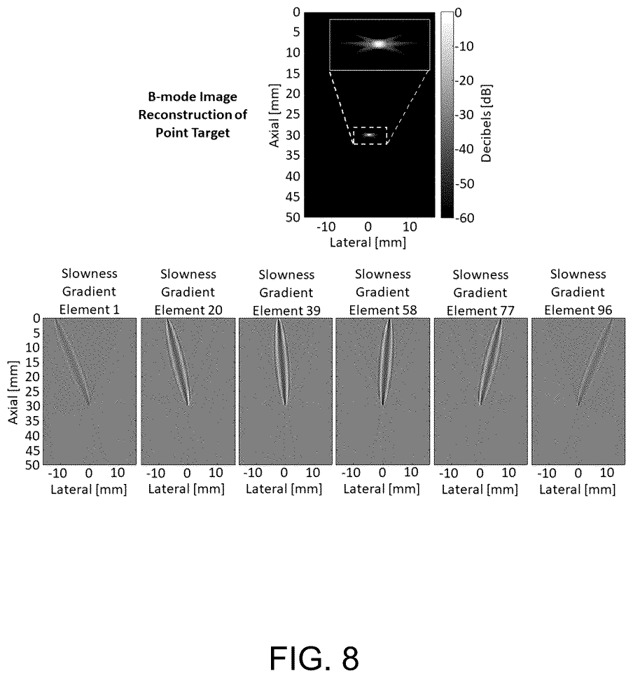

values along “wave-paths” (see FIG. 8). Our basic description of WEMVA could also be modified to incorporate common midpoint gathers, source encoding, etc. These variations ultimately represent alternative ways to compute

$${b}_{l} ( {\vec{x}}_{k}^{im} ) .$$

FIG. 8 depicts slowness gradients for the pulse-echo reconstructions of a point target. The images in FIG. 8 show a point target image and wave paths induced between each transmit element and the point target when the image is focused at a sound speed cbfn equal to the ground-truth sound speed ctrue=1540 m/s in the medium.

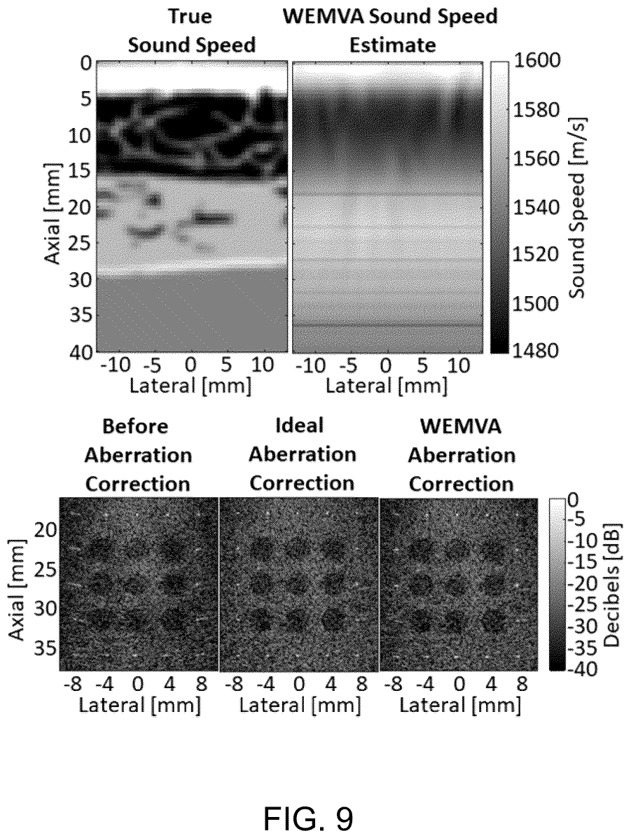

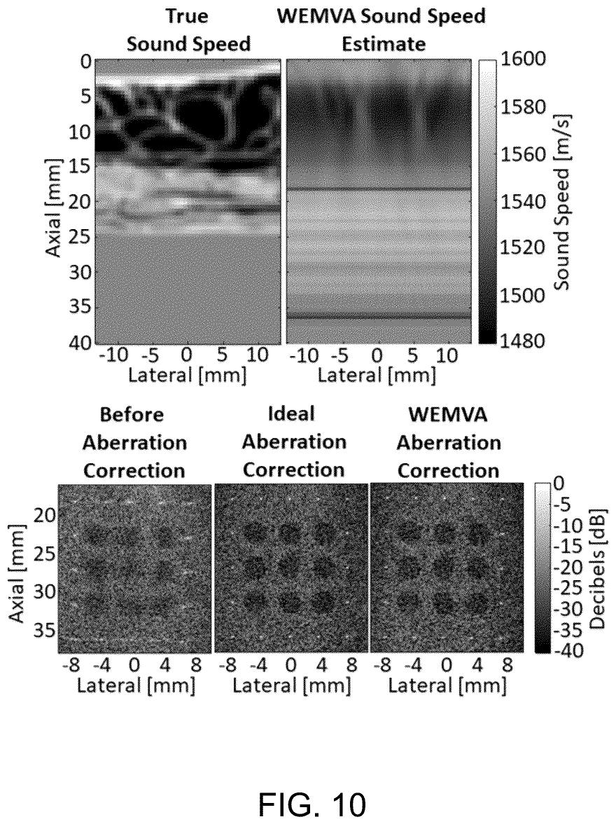

FIGS. 9 and 10 depict ray-based WEMVA in two different simulated abdominal imaging cases. In each case, the B-mode images are shown before aberration correction, after the ideal correction using the true sound speed profile, and corrections using the sound speed reconstructed by ray-based WEMVA. Regularization is to promote a layered structure in the corresponding sound speed reconstructions.

The mathematical descriptions provided above, as known to those skilled in the art, can be implemented in software that is then resident on a computing device or system that has a processor, memory, and access to computer readable media. Such systems have been previously described herein, and provide for an improved ultrasound image that has heretofore not been possible.

It is, therefore, apparent that there has been provided, in accordance with the various objects of the present invention, a Diffraction Tomography System for Sound Speed Estimation and Aberration Correction in Medical Pulse-Echo Ultrasound Imaging Based on Migration Velocity Analysis.

While the various objects of this invention have been described in conjunction with preferred embodiments thereof, it is evident that many alternatives, modifications, and variations will be apparent to those skilled in the art. Accordingly, it is intended to embrace all such alternatives, modifications and variations that fall within the spirit and broad scope of this specification, drawings and claims appended herein.

What is claimed is:

1. An improved system for ultrasound imaging comprising:
an ultrasound system comprising a sending subsystem and a data acquisition subsystem;
an ultrasound probe operatively connected to the ultrasound system;
a wave equation migration velocity analysis (WEMVA) based signal processing component that provides a reflectivity image and a sound speed map; and
an output device configured to receive a reflectivity image and a sound speed map.

2. The system of claim 1, wherein the wave equation migration velocity analysis (WEMVA) based signal processing component comprises a shot-profile migration algorithm, a wave equation migration velocity analysis (WEMVA) algorithm, and a sound speed map output from the wave equation migration velocity analysis (WEMVA) algorithm and input to the shot-profile migration algorithm.

3. The system of claim 2, wherein the wave equation migration velocity analysis (WEMVA) based signal processing component further comprises a coherent compounding algorithm configured to receive reconstructed images from the shot-profile migration algorithm and generate a reflectivity image.

4. The system of any one of claims 1-3, wherein the wave equation migration velocity analysis (WEMVA) algorithm is ray based.

5. The system of any one of claims 1-4, wherein the output device is selected from the group consisting of a display, a digital storage device, or a printer.

6. The system of any one of claims 1-5, further comprising a network interface operatively connected to the output device.

7. The system of claim 1, wherein the wave equation migration velocity analysis (WEMVA) based signal processing component is configured to receive and process received data and send metadata from the ultrasound system.

8. A wave equation migration velocity analysis based (WEMVA) signal processing system comprising:
a shot-profile migration algorithm, a wave equation migration velocity analysis (WEMVA) algorithm, and a sound speed map output from the wave equation migration velocity analysis (WEMVA) algorithm and input to the shot-profile migration algorithm.

9. The wave equation migration velocity analysis (WEMVA) based signal processing system of claim 8, wherein the wave equation migration velocity analysis (WEMVA) based signal processing system further comprises a coherent compounding algorithm configured to receive reconstructed images from the shot-profile migration algorithm and generate a reflectivity image.

10. The wave equation migration velocity analysis (WEMVA) based signal processing system of claim 8 or 9, wherein the wave equation migration velocity analysis (WEMVA) algorithm is ray based.

11. A method for iterative image refinement and sound speed tissue characterization, the method comprising the steps of:
acquiring medical ultrasound pulsed-echo data from an ultrasound data acquisition system;
applying a shot-profile migration algorithm to reconstruct images from the acquired data;
applying a wave equation migration velocity analysis (WEMVA) algorithm to the reconstructed images to update a sound speed map; and
providing updated sound speed map data to the shot-profile migration algorithm.

12. The method of claim 11, further comprising the step of:
providing the updated sound speed map to an output device.

13. The method of claim 11, further comprising the steps of:
providing reconstructed images from the shot-profile migration algorithm to a coherent compounding algorithm; and
generating a reflectivity image from the reconstructed images provided to the shot-profile migration algorithm.

14. The method of claim 13, further comprising the step of:
providing the reflectivity image to an output device.

15. The method of claim 14, further comprising the step of:
providing the updated sound speed map and the reflectivity image to an output device.

16. The method of claim 15, wherein the output device is connected to a network interface.

17. The method of claim 15, wherein the output device is selected from the group consisting of a display, a digital storage device, or a printer.

18. The method of claim 11, wherein the medical ultrasound pulsed-echo data comprises receive data and send metadata.

19. The method of claim 11, further comprising the step of:
providing the updated sound speed map and the reflectivity image to a network.

20. The method of claim 11, wherein the wave equation migration velocity analysis (WEMVA) algorithm is ray based.
